(19) United States
(12) Patent Application Publication
(10) Pub. No.: US 2022/0111288 A1
(43) Pub. Date: Apr. 14, 2022

(54) APPARATUS HAVING BIOMETRIC SENSORS

(71) Applicant: Hewlett-Packard Development Company, L.P., Spring, TX (US)

(72) Inventors: Sarthak Ghosh, Palo Alto, CA (US); Mithra Vankipuram, Palo Alto, CA (US)

(73) Assignee: Hewlett-Packard Development Company, L.P., Spring, TX (US)

(21) Appl. No.: 17/431,761

(22) PCT Filed: Jun. 12, 2019
(86) PCT No.: PCT/US2019/036665

§ 371 Date: Aug. 18, 2021

(51) Int. Cl. A63F 13/212; A63F 13/213; A63F 13/2145; A63F 13/24; A61B 5/024; G06F 3/01

(52) U.S. Cl. CPC A63F 13/212; A63F 13/213; A63F 13/2145; A63F 2300/8082; A61B 5/02427; G06F 3/011; A63F 2300/1012; A63F 13/24

(57) ABSTRACT

In some examples, an apparatus such as an extended reality device can include a handle, an input mechanism, and a biometric sensor located on the input mechanism, where the biometric sensor is to be in contact with an outer surface of a user of the apparatus to measure a physiological state of the user.

### BACKGROUND

Extended reality (XR) devices may be used to provide an altered reality to a user. An XR device may include a virtual reality (VR) device, a mixed reality (MR) device, and/or an augmented reality (AR) device, XR devices may include displays to provide a “virtual and/or augmented” reality experience to the user by providing video, images, and/or other visual stimuli to the user via the displays. XR devices may include audio output devices to provide audible stimuli to the user to further the virtual reality experienced by the user. XR devices may include hand-held XR devices to supplement the extended reality experience by a user. For example, a hand-held XR device may be used to virtually simulate hand motions by the user, such as movement, grasping, releasing, etc.

### BRIEF DESCRIPTION OF THE DRAWINGS

FIG. 1 is a side view of an example of an apparatus having a biometric sensor consistent with the disclosure.

FIG. 2 is a side view of an example of an XR device having input mechanisms and PPG sensors consistent with the disclosure.

FIG. 3 is a side view of an example of a hand-held XR controller having input mechanisms and PPG sensors and a controller consistent with the disclosure.

### DETAILED DESCRIPTION

XR devices may provide an altered reality to a user by providing video, audio, images, and/or other stimuli to a user via a display. As used herein, the term “XR device” refers to a device that provides a virtual, mixed, and/or augmented reality experience for a user.

The XR device may be experienced by a user through the use of a head mount device (e.g., a headset) and/or a hand-held XR device. For example, a user may wear the headset in order to view the display of the XR device and/or experience audio stimuli of the XR device, and/or utilize the hand-held XR device to virtually simulate hand motions by the user, such as movement, grasping, releasing, etc. As used herein, the term “extended reality” refers to a computing device generated scenario that simulates experience through senses and perception. In some examples, an XR device may cover a user's eyes and provide visual stimuli to the user via a display, thereby substituting an “extended” reality (e.g., a “virtual reality”, a “mixed reality”, and/or an “augmented reality”) for actual reality. In some examples, an XR device may cover a user's ears and provide audible stimuli to the user via audio output devices to enhance the virtual reality experienced by the user. In some examples, an XR device may provide an overlay transparent or semi-transparent screen in front of a user's eyes such that reality is “augmented” with additional information such as graphical representations and/or supplemental data. For example, an XR device may overlay transparent or semi-transparent weather information, directions, and/or other information on an XR display for a user to examine.

A hand-held XR device may be used in conjunction with the XR device and can be a useful way to simulate hand motions by a user. For example, while experiencing an extended reality via a headset, a user can utilize a hand-held XR device to simulate hand motions, which can be simulated and presented for the user via the screen(s) included with the XR headset.

Monitoring physiological data of a user while the user is in an extended reality experience can yield information which can help developers understand a user's emotions and/or cognitive load while the user is in the extended reality experience. Utilizing sensors on a hand-held XR device can allow for this physiological information to be obtained. For example, a sensor in contact with a fingertip of a user can yield physiological data which may be useful for XR developers.

Sensors can be placed on the hand-held XR device so that ergonomics of the XR device do not change. Positioning sensors without changing ergonomics of the XR device can allow for the sensors to maintain proper contact with a user at a proper location on the user so that accurate physiological data can be obtained.

An apparatus having biometric sensors, according to the disclosure, can allow for a hand-held XR device to ensure sufficient contact between a user and a sensor included on the hand-held XR device to yield physiological data from the user. Positioning sensors without changing the ergonomics of the hand-held XR device can allow for areas of a user, such as the user's fingertips, to naturally rest on the sensors of the hand-held XR device. In this manner, the sensor can maintain sufficient contact with a particular area of the user so that accurate physiological data describing the user's extended reality experience can be obtained.

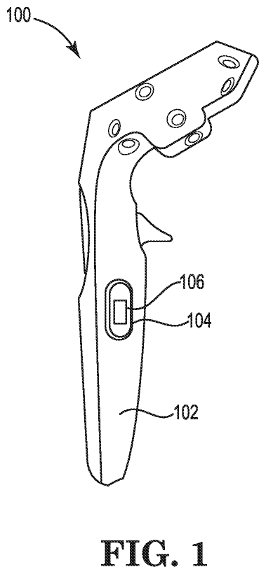

FIG. 1 is a side view of an example of an apparatus 100 having a biometric sensor consistent with the disclosure. Apparatus 100 can include a handle 102, an input mechanism 104, and a biometric sensor 106.

As illustrated in FIG. 1, apparatus 100 can include a handle 102. As used herein, the term “handle” refers to a member which can be grasped or held by a hand of a user. For example, a user may interact with apparatus 100 by grasping and/or holding the handle 102 with their hand.

The handle 102 can be ergonomically shaped. As used herein, the term “ergonomics” refers to a design principle focused around interaction with a human form. For example, the handle 102 can be shaped such that a user's hand can naturally grasp or hold the handle 102 based on a natural shape of the user's hand. The ergonomically shaped handle 102 can allow for a user to grasp and/or hold the apparatus 100 via the handle 102 for extended periods of time without fatigue or stress on the user.

The apparatus 100 can include an input mechanism 104. As used herein, the term “input mechanism” refers to a device by which the apparatus 100 can receive an input from a user. A user can press the input mechanism 104 to cause an input to the apparatus 100. For example, a user can press the input mechanism 104 to simulate a grasping or other hand motion in the extended reality experience, among other examples. As illustrated in FIG. 1, the input mechanism 104 can be located on the handle 102.

The apparatus 100 can include a biometric sensor 106. As used herein, the term “biometric sensor” refers to a device to detect events and/or changes in its environment and transmit the detected events and/or changes for processing and/or analysis. The biometric sensor 106 can detect events and/or changes related to a person based on a physiological and/or behavior characteristic. For example, the biometric sensor 106 can detect events/changes related to the user holding the apparatus 100, as is further described herein.

In some examples, the biometric sensor 106 can be a photoplethysmography (PPG) sensor. As used herein, the term “PPG sensor” refers to a sensor which measures blood volume changes in a bed of tissue. For example, a PPG sensor can detect blood volume changes in a microvascular bed of tissue. Detecting blood volume changes in a microvascular bed of tissue can allow the biometric sensor 106 to measure a physiological state of a user interacting with the apparatus 100, as is further described herein.

As described above, a user may be using the apparatus 100 by grasping and/or holding apparatus 100 via the handle 102. As a result of the user holding the apparatus 100 via the handle 102, the user's fingertip can, in some instances, be placed on the biometric sensor 106.

Accordingly, the biometric sensor 106 can be in contact with an outer surface of the user (e.g., the user's fingertip) such that the apparatus 100 can measure a physiological state of the user. As used herein, the term “physiological state” refers to a condition of a body of a user. For example, the physiological state of the user can be a biometric signal measured by the biometric sensor 106 that can communicate biometric information (e.g., physiological data) about the user interacting with the apparatus 100, as is further described herein.

As described above, the outer surface of the user can be a fingertip of the user. As such, the biometric sensor 106 can be in contact with the fingertip of the user. The fingertip of the user can communicate a physiological state to the apparatus 100 via the biometric sensor 106.

The biometric signal of the user can be a heart rate of the user. For example, as previously described above, the biometric sensor 106 can be a PPG sensor which can measure blood volume changes in a bed of tissue. Detecting blood volume changes in a microvascular bed of tissue can allow for a heart rate of the user to be determined, Heart rate, as a signal, can be used to determine a user's emotional response to extended reality content. Additionally, it can also be used to determine states of relaxation in the user or mental workload a user may be experiencing while in extended reality, among other examples.

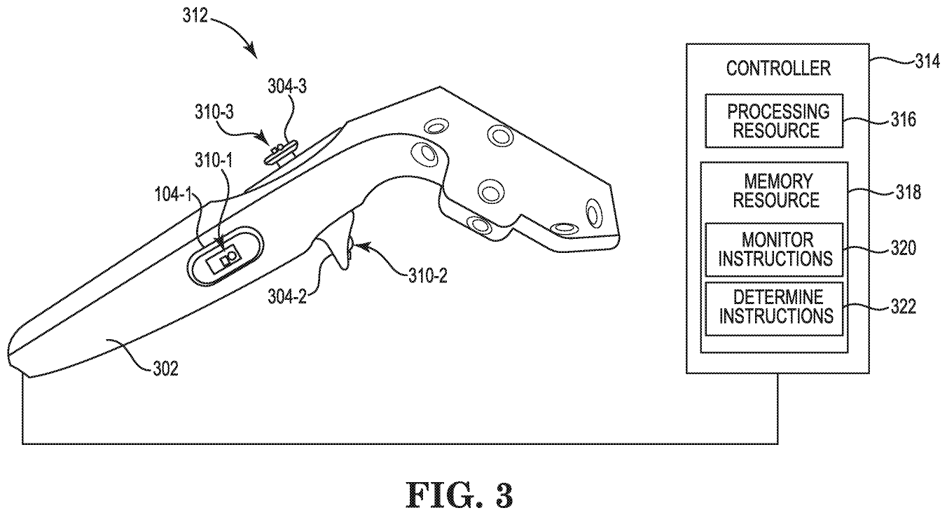

In some examples, the physiological state of the user can be a touch signal. For example, the biometric sensor 106 can be a PPG sensor which can determine whether a user is touching the PPG sensor. Using such information can allow a controller (e.g., controller 314, described in connection with FIG. 3) to determine when a user is holding the apparatus 100.

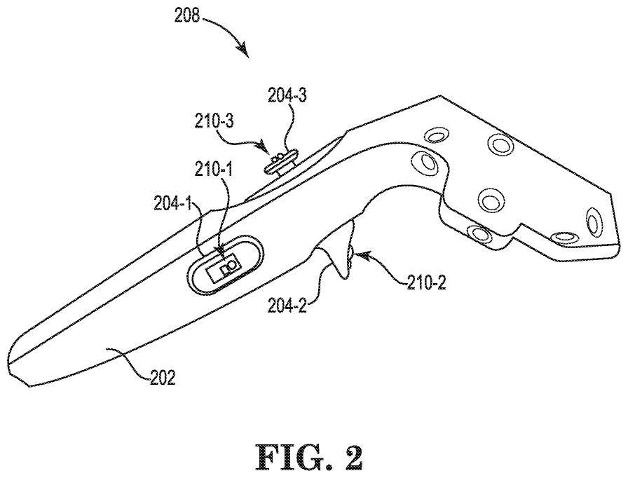

Although the apparatus 100 is described above as including one input mechanism 104 having a biometric sensor 106, examples of the disclosure are not so limited. For example, the apparatus 100 can include more than one input mechanism 104 and more than one biometric sensor 106, as is further described in connection with FIGS. 2 and 3.

FIG. 2 is a side view of an example of an XR device 208 having input mechanisms 204 and PPG sensors 210 consistent with the disclosure. The XR device 208 can include a handle 202, input mechanisms 204-1, 204-2, 204-3 (referred to collectively herein as input mechanisms 204), and PPG sensors 210-1, 210-2, 210-3 (referred to collectively herein as PPG sensors 210).

As previously described in connection with FIG. 1, the XR device 208 can include a handle 202. The handle 202 can include input mechanisms 204. For example, the handle 202 can include an input mechanism 204-1 located on the side of handle 202, an input mechanism 204-2 located on a bottom of the handle 202, and an input mechanism 204-3 located on a top of the handle 202, as oriented in FIG. 2. The input mechanisms 204-1, 204-2, and 204-3 can be located on the handle 202 such that a user grasping handle 202 can easily orient their fingertip(s) to the locations of input mechanisms 204-1, 204-2, 204-3.

As illustrated in FIG. 2, each of the input mechanisms 204-1, 204-2, 204-3 can include a PPG sensor. PPG sensors 210-1, 210-2, 210-3 can be in contact with an outer surface of the user (e.g., the user's fingertip(s)) of the XR device 208. For example, a user resting their fingertip on any one of input mechanisms 204-1, 204-2, 204-3 can contact PPG sensors 210-1, 210-2, 210-3, respectively, such that the PPG sensors 210 can measure blood volume changes in the user's fingertip(s). The measured changes in blood volume of the user can be used to determine a heart rate of the user and/or whether the user is touching the XR device 208, as is further described herein.

Although PPG sensors 210-1, 210-2, 210-3 are described above as being included on each one of the input mechanisms 204-1, 204-2, 204-3, respectively, examples of the disclosure are not so limited. For example, input mechanism 204-1 and 204-3 can include a PPG sensor whereas input mechanism 204-2 does not, input mechanisms 204-2 and 204-3 can include a PPG sensor whereas input mechanism 204-1 does not, etc. In other words, the XR device 208 can include input mechanisms 204 which may not necessarily all include a PPG sensor 210.

The XR device 208 can include the input mechanism 204-1, where the input mechanism 204-1 is a button input mechanism. As used herein, the term “button” refers to a depressible mechanical element to trigger an event and reports depression of the mechanical element to a device. For example, the button input mechanism can receive an input from a user of the XR device 208 in response to the button input mechanism being depressed by a user and reports depression of the button input mechanism to a controller (e,g., controller 314, further described in connection with FIG. 3). For example, the user can depress the button input mechanism to simulate a grasping or other hand motion in the extended reality experience, among other examples, As illustrated in FIG. 2, the button input mechanism (e.g., input mechanism 204-1) can include a PPG sensor 210-1 located thereon. Accordingly, the PPG sensor 210-1 can detect a heart rate of the user and/or whether the user is touching the PPG sensor 210-1, as described above.

The XR device 208 can include the input mechanism 204-2, where the input mechanism 204-2 is a trigger input mechanism. As used herein, the term “trigger” refers to a depressible mechanical lever element to trigger an event and reports depression of the mechanical element to a device. For example, the trigger input mechanism can receive an input from a user of the XR device 208 in response to the trigger input mechanism being depressed by a user and reports depression of the trigger input mechanism to a controller (e.g., controller 314, further described in connection with FIG. 3). For example, the user can depress the trigger input mechanism to simulate a grasping or other hand motion in the extended reality experience, among other examples. As illustrated in FIG. 2, the trigger input mechanism (e.g., input mechanism 204-2) can include a PPG sensor 210-2 located thereon. Accordingly, the PPG sensor 210-2 can detect a heart rate of the user and/or whether the user is touching the PPG sensor 210-2, as described above.

The XR device 208 can include the input mechanism 204-3, where the input mechanism 204-3 is a joystick input mechanism. As used herein, the term “joystick” refers to a shaft that pivots on a base and reports its angle and/or direction to a controller (e.g., controller 314, further described in connection with FIG. 3). For example, the joystick input mechanism can receive an input from a user of the XR device 208 in response to the joystick input mechanism being pivoted by an angle and/or a direction by a user. For example, the user can move the joystick input mechanism to simulate a grasping or other hand motion in the extended reality experience, among other examples. As illustrated in FIG. 2, the joystick input mechanism (e.g., input mechanism 204-3) can include a PPG sensor 210-3 located thereon. Accordingly, the PPG sensor 210-3 can detect a heart rate of the user and/or whether the user is touching the PPG sensor 210-3, as described above.

Although the XR device 208 is illustrated in FIG. 2 as including the input mechanism 204-1, 204-2, and 204-3, as well as the PPG sensors 210-1, 210-2, and 210-3, examples of the disclosure are not so limited. For example, the XR device 208 can include any subset of the input mechanisms 204 (e.g., the XR device 208 can include the input mechanisms 204-1 and 204-2, 204-1 and 204-3, 204-2 and 204-3, 204-1, 204-2, or 204-3, and/or any other combinations thereof). Additionally, the XR device 208 can include any subset of the PPG sensors 210-1, 210-2, 210-3 (e.g., the PPG sensors 210-1 and 210-2 when the XR device 208 includes input mechanisms 204-1 and 204-2, the PPG sensors 210-1 and 210-3 when the XR device 208 includes input mechanisms 204-1 and 204-3, etc.).

FIG. 3 is a side view of an example of a hand-held XR controller 312 having input mechanisms 304 and PPG sensors 310 and a controller 314 consistent with the disclosure. The hand-held XR controller 312 can include a handle 302, input mechanisms 304-1, 304-2, 304-3 (referred to collectively herein as input mechanisms 304), and PPG sensors 310-1, 310-2, 310-3 (referred to collectively herein as PPG sensors 310).

The hand-held XR controller 312 can include a handle 302. The handle 302 can include input mechanisms 304. For example, the handle 302 can include an input mechanism 304-1 located on the side of handle 202, an input mechanism 304-2 located on a bottom of the handle 302, and an input mechanism 304-3 located on a top of the handle 302, as oriented in FIG. 3. The input mechanisms 304-1, 304-2, and 304-3 can be located on the handle 302 such that a user grasping handle 302 can easily orient their fingertip(s) to the locations of input mechanisms 304-1, 304-2, 304-3.

In some examples, the hand-held XR controller 312 can include input mechanisms 304-1 including a button. For example, the button can receive an input from a user of the hand-held XR controller 312 in response to the user depressing the button. Depression of the button can simulate a grasping or other hand motion in the extended reality experience, among other examples.

In some examples, the hand-held XR controller 312 can include input mechanisms 304-2 including a trigger. For example, the trigger can receive an input from a user of the hand-held XR controller 312 in response to the user depressing the trigger. Depression of the trigger can simulate a grasping or other hand motion in the extended reality experience, among other examples.

In some examples, the hand-held XR controller 312 can include input mechanisms 304-3 including a joystick. For example, the joystick can receive an input from a user of the hand-held XR controller 312 in response to the user pivoting the joystick to a particular angle and/or direction. Pivoting of the joystick can simulate a grasping or other hand motion in the extended reality experience, among other examples.

As illustrated in FIG. 3, each of the input mechanisms 304-1, 304-2, 304-3 can include a PPG sensor 310. PPG sensors 310-1, 310-2, 310-3 can be in contact with at least one outer surface of the user (e.g., the user's fingertip(s)). For example, a user resting their fingertip on any one of input mechanisms 304-1, 304-2, 304-3 can contact PPG sensors 310-1, 310-2, 310-3, respectively, such that the PPG sensors 310 can measure a physiological state from the user. The physiological state can be a biometric signal such as a heart rate and/or whether a user is touching the PPG sensors 310-1, 310-2, 310-3, as is further described herein.

Although the hand-held XR controller 312 is illustrated in FIG. 2 as including the input mechanism 304-1, 304-2, and 304-3, as well as the PPG sensors 310-1, 310-2, and 310-3, examples of the disclosure are not so limited. For example, the hand-held XR controller 312 can include any subset of the input mechanisms 304 (e.g., the hand-held XR controller 312 can include the input mechanisms 304-1 and 304-2, 304-1 and 304-3, 304-2 and 304-3, 304-1, 304-2, or 304-3, and/or any other combinations thereof). Additionally, the hand-held XR controller 312 can include any subset of the PPG sensors 310-1, 310-2, 310-3 (e.g., the PPG sensors 310-1 and 310-2 when the hand-held XR controller 312 includes input mechanisms 304-1 and 304-2, the PPG sensors 310-1 and 310-3 when the hand-held XR controller 312 includes input mechanisms 304-1 and 304-3, etc.).

In some examples, the hand-held XR controller 312 can be connected to a controller 314. As described herein, the controller 314 may perform functions related to an apparatus having biometric sensors. Although not illustrated in FIG. 3, the controller 314 may include a processor and a machine-readable storage medium. Although the following descriptions refer to a single processor and a single machine-readable storage medium, the descriptions may also apply to a system with multiple processors and multiple machine-readable storage mediums. In such examples, the controller 314 may be distributed across multiple machine-readable storage mediums and the controller 430 may be distributed across multiple processors. Put another way, the instructions executed by the controller 314 may be stored across multiple machine-readable storage mediums and executed across multiple processors, such as in a distributed or virtual computing environment.

The processing resource 316 may be a central processing unit (CPU), a semiconductor based microprocessor, and/or other hardware devices suitable for retrieval and execution of machine-readable instructions 320, 322 stored in a memory resource 318. Processing resource 316 may fetch, decode, and execute instructions 320, 322. As an alternative or in addition to retrieving and executing instructions 320, 322, processing resource 316 may include a plurality of electronic circuits that include electronic components for performing the functionality of instructions 320, 322.

Memory resource 318 may be any electronic, magnetic, optical, or other physical storage device that stores executable instructions 320, 322 and/or data. Thus, memory resource 318 may be, for example, Random Access Memory (RAM), an Electrically-Erasable Programmable Read-Only Memory (EEPROM), a storage drive, an optical disc, and the like. Memory resource 318 may be disposed within controller 314, as shown in FIG. 3. Additionally and/or alternatively, memory resource 318 may be a portable, external or remote storage medium, for example, that causes controller 314 to download the instructions 320, 322 from the portable/external/remote storage medium.

The controller 314 may include instructions 320 stored in the memory resource 318 and executable by the processing resource 316 to monitor the PPG sensors 310 located on each one of the input mechanisms 304. For example, the controller 314 can monitor the PPG sensors 310-1, 310-2, 310-3 located on the input mechanisms 304-1, 304-2, 304-3, respectively, for blood volume changes in the user's fingertip(s) contacting the PPG sensors 310-1, 310-2, 310-3.

The controller 314 may include instructions 320 stored in the memory resource 318 and executable by the processing resource 316 to determine a physiological state of a user based on the monitored PPG sensors 310. The physiological state of the user can be a biometric signal. For example, the controller 314 can monitor the PPG sensors 310-1, 310-2, 310-3 for blood volume changes in the user's fingertip(s). Monitoring the PPG sensors 310 for blood volume changes can allow for a heart rate of the user to be determined. For example, the measured changes in blood volume of the user can be used to determine a heart rate of the user. In some examples, the controller 314 can monitor the PPG sensors 310-1, 310-2, 310-3 to determine whether a user is touching the PPG sensors 310-1, 310-2, 310-3 (e.g., determine a touch signal). Accordingly, the controller 314 can generate, modify, and/or adjust an animation (e.g., experienced by the user) in the extended reality experience based on the determined physiological state of the user. As used herein, the term “animation” refers to a dynamic visual medium produced from sequenced images that are manipulated to appear as motion. For instance, generating, modifying, and/or adjusting an animation may be done based on a physiological state of a user. For example, the controller 314 may generate, speed up, and/or slow down animations based on the physiological state of the user, among other examples.

As illustrated in FIG. 3, the hand-held XR controller 312 can include a button input mechanism 304-1, a trigger input mechanism 304-2, and a joystick input mechanism 304-3, where each one of the input mechanisms 304 can include a PPG sensor 310. For example, the button input mechanism 304-1 can include a PPG sensor 310-1, the trigger input mechanism 304-2 can include a PPG sensor 310-2, and the joystick input mechanism 304-3 can include a PPG sensor 310-3. Accordingly, in some examples, the controller 314 can receive signals from each of the PPG sensors 310 located on the input mechanisms 304 as is further described herein.

For example, the controller 314 can include a biometric signal from PPG sensor 310-1, a biometric signal from PPG sensor 310-2, and/or a biometric signal from PPG sensor 310-3. The biometric signal can be, for instance, a heart rate of the user and/or a touch signal from the user.

Receiving multiple biometric signals from the PPG sensors 310 can be useful such that the controller 314 has redundant biometric signals to utilize. However, the controller 314 may have to determine which of the multiple biometric signals from the PPG sensors 310 to utilize.

Accordingly, the controller 314 can include instructions stored in the memory resource 318 and executable by the processing resource 316 to compare the plurality of signals. For example, the controller 314 can execute instructions to compare the biometric signals received from the PPG sensors 310 via wavelet analysis. As used herein, the term “wavelet” refers to a signal having a wave-like oscillation with an amplitude that begins at zero, increases, and then decreases back to zero. As used herein, the term “wavelet analysis” refers to decomposing a signal into lower resolution levels by controlling scaling and shifting factors of a single wavelet function. For example, the controller 314 can decompose various signals received from PPG sensors 310 by controlling scaling and shifting factors.

The controller 314 can include instructions stored in the memory resource 318 and executable by the processing resource 316 to determine a heart rate of the user based on a particular physiological state from the signals received from the PPG sensors 310 which satisfies a threshold condition. For example, the controller 314 can receive a first biometric signal from the PPG sensor 310-1 located on the button input mechanism 304-1, a second biometric signal from the PPG sensor 310-2 located on the trigger input mechanism 304-2, and a third biometric signal from the PPG sensor 310-3 located on the joystick input mechanism 304-3. After performing wavelet analysis on the first, second, and third biometric signals, the controller can determine which of the biometric signals satisfies a threshold condition. The threshold condition can be, for example, a signal quality, a particular signal shape (e.g., a heartbeat shape), among other types of threshold conditions. Accordingly, the controller can determine a heart rate of the user using, for example, the second biometric signal from the PPG sensor 310-2 located on the trigger input mechanism 304-2 based on the second biometric signal having a signal quality which exceeds a threshold signal quality. As another example, the controller can determine a heart rate of the user using, for example, the third biometric signal from the PPG sensor 310-3 located on the joystick input mechanism 304-3 based on the third biometric signal exceeding a threshold signal shape of a heartbeat shape.

The controller 314 can include instructions stored in the memory resource 318 and executable by the processing resource 316 to determine that the user is not touching an input mechanism of the input mechanisms 304. For example, in response to the PPG sensors 310 not detecting a physiological state of the user, the controller 314 can determine that the user is not touching any of the input mechanisms 304. For instance, if a user is not touching PPG sensors 310-1, 310-2, 310-3, the controller 314 can determine that the user is not touching any of the input mechanisms 304-1, 304-2, 304-3. Accordingly, the controller 314 can prevent animation of a grasping or other hand motion in the extended reality experience, among other examples.

An apparatus having biometric sensors, according to the disclosure, can allow an XR device to ensure sufficient contact between a user and a biometric sensor. Ensuring sufficient contact between the user and the biometric sensor can allow for accurate physiological data, such as whether the user is touching an input mechanism and/or a heart rate of the user, describing a user's extended reality experience to be obtained in order to accurately simulate motion in the extended reality experience while allowing the user to grasp and/or hold the XR device for extended periods of time without fatigue or stress on the user.

In the foregoing detailed description of the disclosure, reference is made to the accompanying drawings that form a part hereof, and in which is shown by way of illustration how examples of the disclosure may be practiced. These examples are described in sufficient detail to enable those of ordinary skill in the art to practice the examples of this disclosure, and it is to be understood that other examples may be utilized and that process, electrical, and/or structural changes may be made without departing from the scope of the disclosure. Further, as used herein, “a” can refer to one such thing or more than one such thing.

The figures herein follow a numbering convention in which the first digit corresponds to the drawing figure number and the remaining digits identify an element or component in the drawing. For example, reference numeral 102 may refer to element 102 in FIG. 1 and an analogous element may be identified by reference numeral 202 in FIG. 2. Elements shown in the various figures herein can be added, exchanged, and/or eliminated to provide additional examples of the disclosure. In addition, the proportion and the relative scale of the elements provided in the figures are intended to illustrate the examples of the disclosure, and should not be taken in a limiting sense.

It can be understood that when an element is referred to as being “on,” “connected to”, “coupled to”, or “coupled with” another element, it can be directly on, connected, or coupled with the other element or intervening elements may be present. In contrast, when an object is “directly coupled to” or “directly coupled with” another element it is understood that are no intervening elements (adhesives, screws, other elements) etc.

The above specification, examples and data provide a description of the method and applications, and use of the system and method of the disclosure. Since many examples can be made without departing from the scope of the system and method of the disclosure, this specification merely sets forth some of the many possible example configurations and implementations.

What is claimed is:

1. An apparatus, comprising:
a handle;
an input mechanism; and
a biometric sensor located on the input mechanism;
wherein the biometric sensor is to be in contact with an outer surface of a user of the apparatus to measure a physiological state of the user.

2. The apparatus of claim 1, wherein the biometric sensor is a photoplethysmography (PPG) sensor.

3. The apparatus of claim 1, wherein the physiological state of the user is a biometric signal of the user.

4. The apparatus of claim 3, wherein the biometric signal is a heart rate of the user.

5. The apparatus of claim 1, wherein the physiological state of the user is a touch signal from the user.

6. The apparatus of claim 1, wherein the outer surface of the user is a fingertip of the user such that the biometric sensor is to be in contact with the fingertip of the user.

7. A controller of an extended reality (XR) device, comprising:
a handle;
a plurality of input mechanisms located on the XR device; and
a photoplethysmography (PPG) sensor located on each one of the plurality of input mechanisms;
wherein the PPG sensors are to be in contact with an outer surface of a user of the XR device.

8. The controller of claim 7, wherein:
the plurality of input mechanisms includes a button input mechanism; and
the button input mechanism includes a PPG sensor.

9. The controller of claim 7, wherein:
the plurality of input mechanisms includes a trigger input mechanism; and
the trigger input mechanism includes a PPG sensor.

10. The controller of claim 7, wherein:
the plurality of input mechanisms includes a joystick input mechanism; and
the joystick input mechanism includes a PPG sensor.

11. An extended reality (XR) device, comprising:
a handheld XR controller comprising:
a handle;
a plurality of input mechanisms located on the handheld XR controller; and
a photoplethysmography (PPG) sensor located on each one of the plurality of input mechanisms;
a controller including a memory resource and a processing resource to execute non-transitory machine-readable instructions stored in the memory to:
monitor the PPG sensors located on each one of the plurality of input mechanisms; and
determine a physiological state of a user based on the monitored PPG sensors.

12. The XR device of claim 11, wherein:
the plurality of input mechanisms include a button, a trigger, and a joystick; and
the processing resource executes the instructions to determine a heart rate of the user based on the monitored PPG sensors included on the button, the trigger, and the joystick.

13. The XR device of claim 12, wherein:
the controller receives a plurality of signals from the PPG sensors on each of the plurality of input mechanisms, wherein the plurality of signals include a signal from the PPG sensor included on the button, a signal from the PPG sensor included on the trigger, and a signal from the PPG sensor included on the joystick; and
the processing resource executes the instructions to:
compare the plurality of signals via wavelet analysis; and
determine the heart rate of the user based on a particular signal from the plurality of signals which satisfies a threshold condition.

14. The XR device of claim 11, wherein:
the plurality of input mechanisms include a button, a trigger, and a joystick; and
the processing resource executes the instructions to determine a touch signal from the user based on at least one of the monitored PPG sensors included on the button, the trigger, and the joystick.

15. The XR device of claim 11, wherein the processing resource executes the instructions to determine, in response to the PPG sensors not detecting the physiological state of the user, that the user is not touching an input mechanism of the plurality of input mechanisms.
